(19) United States
(12) Patent Application Publication
(10) Pub. No.: US 2015/0031958 A1
(43) Pub. Date: Jan. 29, 2015

(54) SURGICAL SEAL ASSEMBLY WITH UPPER LIP SEAL

(71) Applicant: Covidien LP, Mansfield, MA (US)

(72) Inventor: Gennady Kleyman, Brooklyn, NY (US)

(73) Assignee: Covidien LP, Mansfield, MA (US)

(21) Appl. No.: 14/286,292

(22) Filed: May 23, 2014

Related U.S. Application Data
(60) Provisional application No. 61/858,222, filed on Jul. 25, 2013.

(51) Int. Cl. A61B 17/34

(52) U.S. Cl. CPC A61B 17/3423; A61B 2017/3445; A61B 2017/3419
USPC 600/204

(57) ABSTRACT

A surgical access device is presented including a seal assembly having an upper housing member and a lower housing member configured to form a housing, the housing defining a central longitudinal axis and having a longitudinal passage for receiving at least one surgical object therethrough. The surgical access device further includes a seal disposed within the seal assembly, the seal movably arranged within the housing. The seal may include an elastomeric seal member having an opening for receiving and sealing with an instrument inserted therethrough, the elastomeric seal member arranged between an upper seal holder and a lower seal holder. The upper seal holder may be a cup-shaped seal having an upper lip that engages at least a portion of the upper housing member to secure the seal within the seal assembly.

### CROSS-REFERENCE TO RELATED APPLICATIONS

This application claims the benefit of and priority to U.S. Provisional Patent Application No. 61/858,222, filed Jul. 25, 2013, the entire disclosure of which is incorporated by reference herein.

### BACKGROUND

1. Technical Field

The present disclosure relates to a surgical access device and, more particularly, but not exclusively, relates to a surgical access device having an upper seal holder with an upper lip configured to engage a seal with an upper housing member to secure the seal within the surgical access device.

2. Background of Related Art

Minimally invasive surgical procedures including both endoscopic and laparoscopic procedures permit surgery to be performed on organs, tissues and vessels far removed from an opening within the tissue. Laparoscopic and endoscopic procedures generally require that any instrumentation inserted into the body be sealed, i.e., provisions must be made to ensure that gases do not enter or exit the body through the incision as, for example, in surgical procedures in which the surgical region is insufflated. These procedures typically employ surgical instruments which are introduced into the body through a cannula. The cannula has a housing at a proximal end thereof in which a seal assembly is mounted. The seal assembly provides a substantially fluid tight seal about the instrument to preserve the integrity of the established pneumoperitoneum.

Minimally invasive procedures have several advantages over traditional open surgery, including less patient trauma, reduced recovery time, reduced potential for infection, etc. However, despite its recent success and overall acceptance as a preferred surgical technique, minimally invasive surgery, such as laparoscopy, has disadvantages. In particular, the insertion of the surgical instrument within the cannula has proven to be difficult in certain procedures, e.g., in procedures requiring extensive manipulation of the long narrow endoscopic instruments within a remote site. In addition, many conventional seal assemblies are not particularly well-adapted to receive a surgical instrument if it is inserted at an angle, thus resulting in damage to the seal assemblies. This type of insertion often results in the instrument missing the target (e.g. septum seal, etc.) and becoming lodged in an undesirable location within the seal assembly. In addition, angulation and/or manipulation of instrumentation within the cannula often present difficulties with respect to maintaining seal integrity. Thus, there remains a need for an apparatus that may be used to guide a surgical instrument through a seal assembly in a more efficient and efficacious manner.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

According to one aspect of the present disclosure, a surgical seal device is provided. The surgical seal device includes a seal assembly having an upper housing member and a lower housing member configured to form a housing, the housing defining a central longitudinal axis and having a longitudinal passage for receiving at least one surgical object therethrough. The surgical access device further includes a seal disposed within the seal assembly, the seal movably arranged within the housing, the seal including an elastomeric seal member having an opening for receiving and sealing with an instrument inserted therethrough, the elastomeric seal member arranged between an upper seal holder and a lower seal holder. The upper seal holder is a cup-shaped seal having an upper lip that engages at least a portion of the upper housing member to secure the seal within the seal assembly.

In one exemplary embodiment, the seal is an elastomeric seal. In another exemplary embodiment, wherein the upper seal holder acts as a guard member for protecting the seal from being damaged by repeated insertion of the at least one surgical instrument therethrough.

In another exemplary embodiment, the lower seal holder includes a plurality of spaced apart and outwardly extending circumferential legs for providing self-centering of the seal.

In yet another exemplary embodiment, the upper housing member defines an angular opening therethrough to facilitate angular reception of the at least one surgical object.

In another exemplary embodiment, the upper seal holder is dimensioned and adapted to prevent passage of fluids through the seal assembly.

In one exemplary embodiment, the upper seal holder includes a channel on a lower end thereof for receiving and securing a distal end of the seal.

In another exemplary embodiment, the upper lip of the upper seal holder includes a plurality of ribs adapted and dimensioned to provide a secure attachment to the upper housing member. The plurality of ribs may be dual circumferential protrusions forming continuous dual concentric circles. Alternatively, the plurality of ribs may be a series of discontinuous circular protrusions.

In yet another exemplary embodiment, the upper lip of the upper seal holder extends beyond the lower seal holder.

According to another aspect of the present disclosure, a cannula assembly is provided. The cannula assembly includes a cannula housing, a cannula sleeve extending distally from the cannula housing and a seal assembly disposed in mechanical cooperation with the cannula housing. The seal assembly includes a seal housing having an upper housing member and a lower housing member, the seal housing defining a central longitudinal axis and having a longitudinal passage for receiving at least one surgical object therethrough. The seal assembly further includes a seal disposed within the seal assembly, the seal movably arranged within the housing, the seal including an elastomeric seal member having an opening for receiving and sealing with an instrument inserted therethrough, the elastomeric seal member arranged between an upper seal holder and a lower seal holder. The upper seal holder is a cup-shaped seal having an upper lip that engages the upper housing member to secure the seal within the seal housing.

In another aspect of the present disclosure, a method for performing a surgical procedure is provided. The method includes the step of providing a seal assembly having an upper housing member and a lower housing member configured to form a housing, the housing defining a central longitudinal axis and having a longitudinal passage for receiving at least one surgical object therethrough. The method further includes the step of providing a seal disposed within the seal assembly, the seal movably arranged within the housing, the seal including an elastomeric seal member having an opening for receiving and sealing with an instrument inserted therethrough, the elastomeric seal member arranged between an upper seal holder and a lower seal holder. The upper seal holder is a cup-shaped seal having an upper lip that engages the upper housing member to secure the seal within the seal assembly.

Further scope of applicability of the present disclosure will become apparent from the detailed description given hereinafter. However, it should be understood that the detailed description and specific examples, while indicating preferred embodiments of the present disclosure, are given by way of illustration only, since various changes and modifications within the spirit and scope of the present disclosure will become apparent to those skilled in the art from this detailed description.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a perspective view of an obturator having a cannula assembly, in accordance with the present disclosure;

FIG. 2 is a side view of the obturator of FIG. 1 illustrating the seal assembly, in accordance with the present disclosure;

FIG. 3 is a perspective view of the obturator with the seal assembly removed from the cannula assembly, in accordance with the present disclosure;

FIG. 4 is a perspective view of the seal assembly, in accordance with an embodiment of the present disclosure;

FIG. 5 is a perspective view of a lower housing member of the seal assembly of FIG. 4, in accordance with the present disclosure;

FIG. 6 is a perspective view of an upper housing member of the seal assembly of FIG. 4, in accordance with the present disclosure;

FIGS. 7 and 8 are top perspective views of the obturator where the tab is in an ON position and in an OFF position, in accordance with the present disclosure;

FIG. 9 is a perspective, cross-sectional view of the seal assembly illustrating the seal having an upper lip engaged with a distal portion of the upper housing member, in accordance with the present disclosure;

FIG. 10 is a side view of the seal assembly illustrating the seal having an upper lip, in accordance with the present disclosure;

FIG. 11 is a perspective view of the seal having an upper lip and a centering mechanism, the upper lip having a plurality of ribs for providing additional securement to the upper housing member, in accordance with the present disclosure;

FIG. 12 is a top perspective view of the seal of FIG. 11, illustrating the upper lip of the seal, as well as the centering mechanism, in accordance with the present disclosure;

FIG. 13 is an exploded view of the seal assembly, illustrating the upper housing member and the lower housing member, as well as the seal disposed therebetween, in accordance with the present disclosure;

FIG. 14 is a top view of the obturator, illustrating the centering mechanism in an unbiased position, in accordance with the present disclosure;

FIG. 15 is a top view of the obturator, illustrating the centering mechanism in a biased position (or off-center location), in accordance with the present disclosure; and

FIG. 16 is a perspective view of the obturator including the seal assembly having the seal with an upper lip, as well as a surgical instrument inserted through the obturator and into tissue, in accordance with the present disclosure.

The figures depict preferred embodiments of the present disclosure for purposes of illustration only. One skilled in the art will readily recognize from the following discussion that alternative embodiments of the structures and methods illustrated herein may be employed without departing from the principles of the present disclosure described herein.

### DETAILED DESCRIPTION

Particular embodiments of the present disclosure are described hereinbelow with reference to the accompanying drawings. However, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

For the purposes of promoting an understanding of the principles of the present disclosure, reference will now be made to the exemplary embodiments illustrated in the drawings, and specific language will be used to describe the same. It will nevertheless be understood that no limitation of the scope of the present disclosure is thereby intended. Any alterations and further modifications of the inventive features illustrated herein, and any additional applications of the principles of the present disclosure as illustrated herein, which would occur to one skilled in the relevant art and having possession of this disclosure, are to be considered within the scope of the present disclosure.

The cannula assembly of the present disclosure, in combination with a seal system internal to the cannula assembly, provides a substantial seal between a body cavity of a patient and the outside atmosphere before, during and after insertion of a surgical instrument through the cannula assembly. Moreover, the seal assembly of the present disclosure is capable of accommodating surgical instruments of varying diameters, e.g., from 5 mm to 15 mm, by providing a fluid tight seal with each instrument when inserted. The flexibility of the present seal assembly greatly facilitates endoscopic surgery where a variety of instruments having differing diameters are often used during a single surgical procedure.

The seal assembly contemplates the introduction and manipulation of various types of instrumentation adapted for insertion through a trocar and/or cannula assembly while maintaining a fluid tight interface about the instrumentation to preserve the atmospheric integrity of a surgical procedure from gas and/or fluid leakage. Specifically, the seal assembly accommodates angular manipulation of the surgical instrument relative to the seal housing axis. This feature of the present disclosure desirably minimizes the entry and exit of gases and/or fluids to/from the body cavity. Examples of instrumentation include clip appliers, graspers, dissectors, retractors, staplers, laser probes, photographic devices, endoscopes and laparoscopes, tubes, and the like. Such instruments will be collectively referred to herein as “instruments or instrumentation.”

The word “exemplary” is used herein to mean “serving as an example, instance, or illustration.” Any embodiment described herein as “exemplary” is not necessarily to be construed as preferred or advantageous over other embodiments. The word “example” may be used interchangeably with the term “exemplary.”

Embodiments of the presently disclosed apparatus will now be described in detail with reference to the drawings, in which like reference numerals designate identical or corresponding elements in each of the several views. As used herein, the term “distal” refers to that portion of the tool, or component thereof which is farther from the user while the term “proximal” refers to that portion of the tool or component thereof which is closer to the user.

Reference will now be made in detail to embodiments of the present disclosure. While certain embodiments of the present disclosure will be described, it will be understood that it is not intended to limit the embodiments of the present disclosure to those described embodiments. To the contrary, reference to embodiments of the present disclosure is intended to cover alternatives, modifications, and equivalents as may be included within the spirit and scope of the embodiments of the present disclosure as defined by the appended claims.

For exemplary purposes, the access apparatus will be described in terms of a cannula assembly, which is adapted for introduction, typically utilizing a trocar, within the abdominal cavity during a laparoscopic surgical procedure. However, it is appreciated that the access apparatus may be any apparatus suitable for introduction and passage of surgical objects into underlying tissue including, e.g., catheters, trocar assemblies, endoscopic portals, hand access devices, etc., through an incision or through a natural body opening.

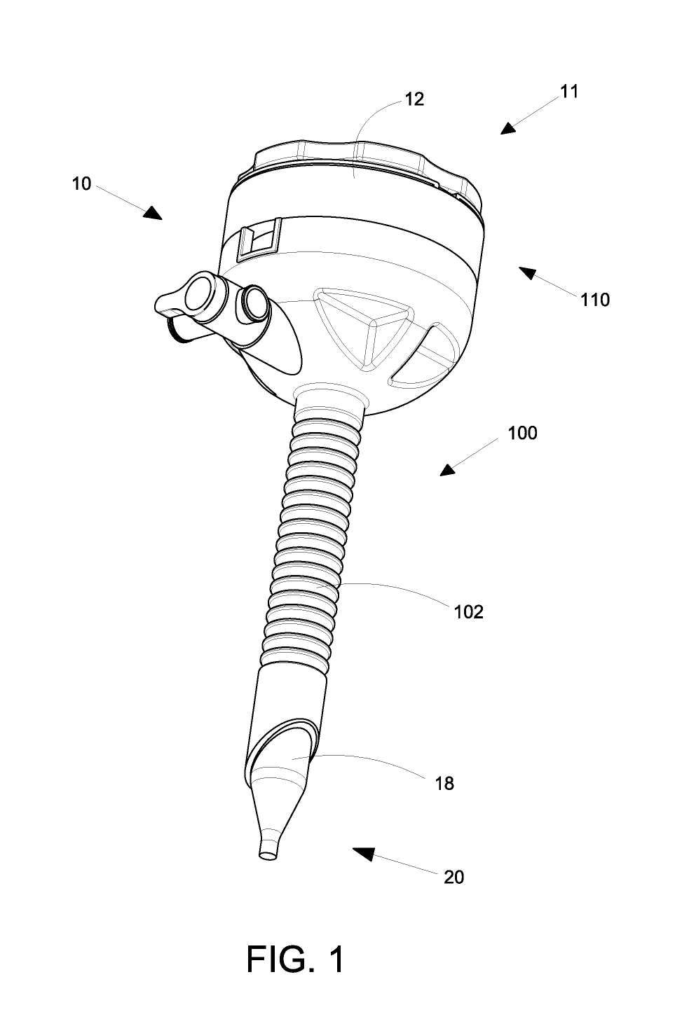

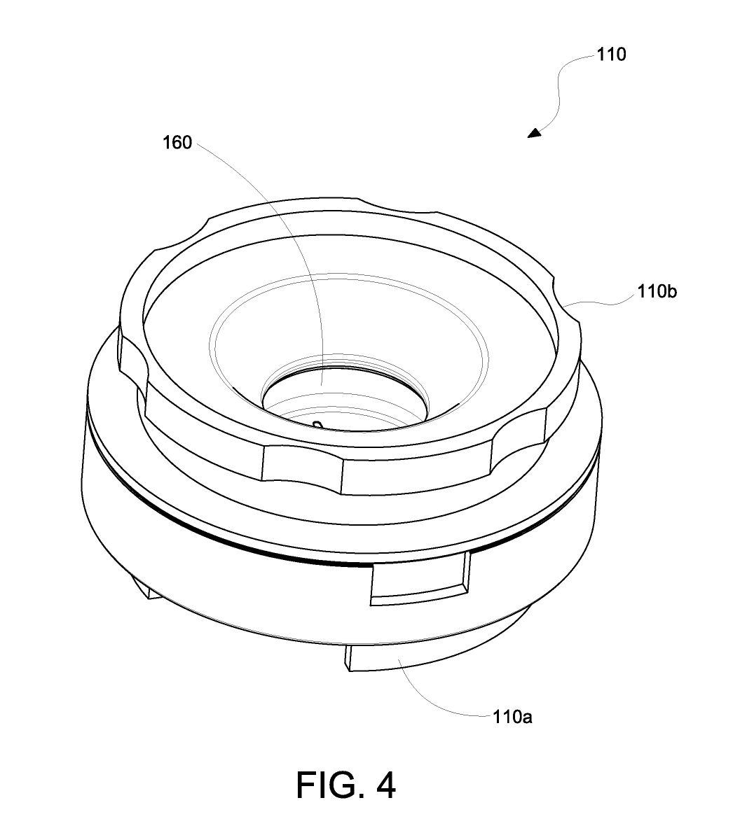

Referring now to the drawings, in which like reference numerals identify identical or substantially similar parts throughout the several views, FIGS. 1-3 illustrate an obturator 10, whereas FIGS. 4-8 illustrate a seal assembly 110.

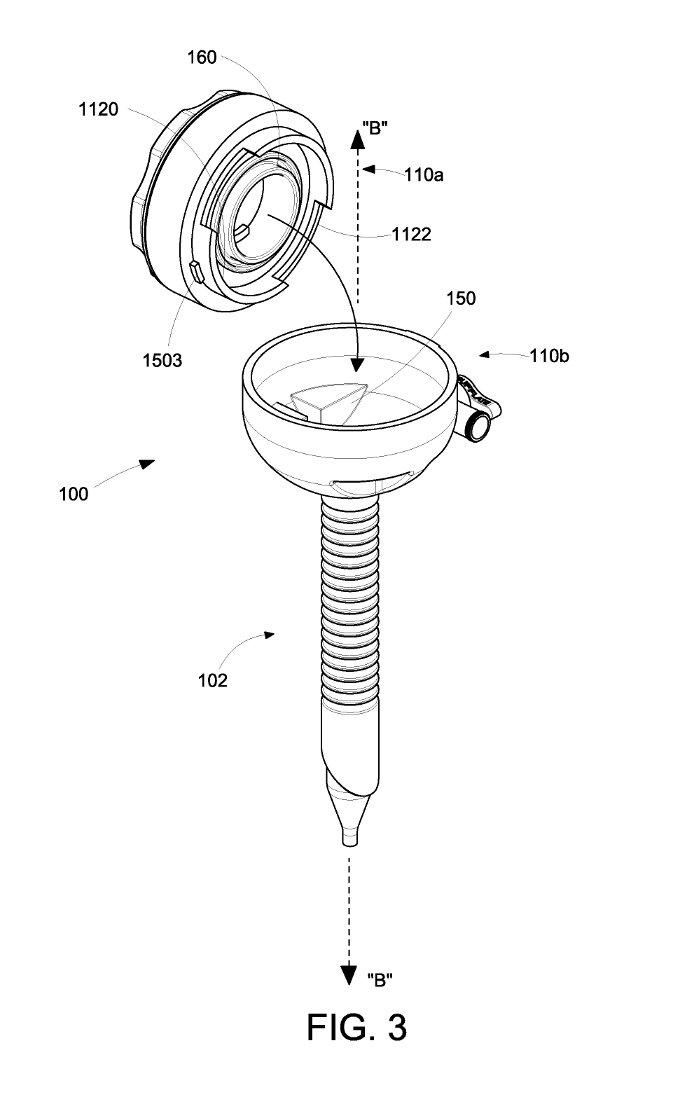

Referring to FIGS. 1-3, the obturator 10 includes an obturator assembly 11 and a cannula assembly 100 which at least partially receives the obturator assembly 11. The obturator assembly 11 includes an obturator housing 12 disposed in mechanical cooperation with an elongated obturator member (not shown), and defines a longitudinal axis “B-B.” (see FIG. 3). An obturator shaft 18 is mechanically coupled to the obturator housing 12 and an optical member 20 is positioned at a distal end of the obturator shaft 18. The obturator shaft 18 may be made from either steel or a polymeric material. One skilled in the art may contemplate using a plurality of different materials to construct the components of the obturator 10.

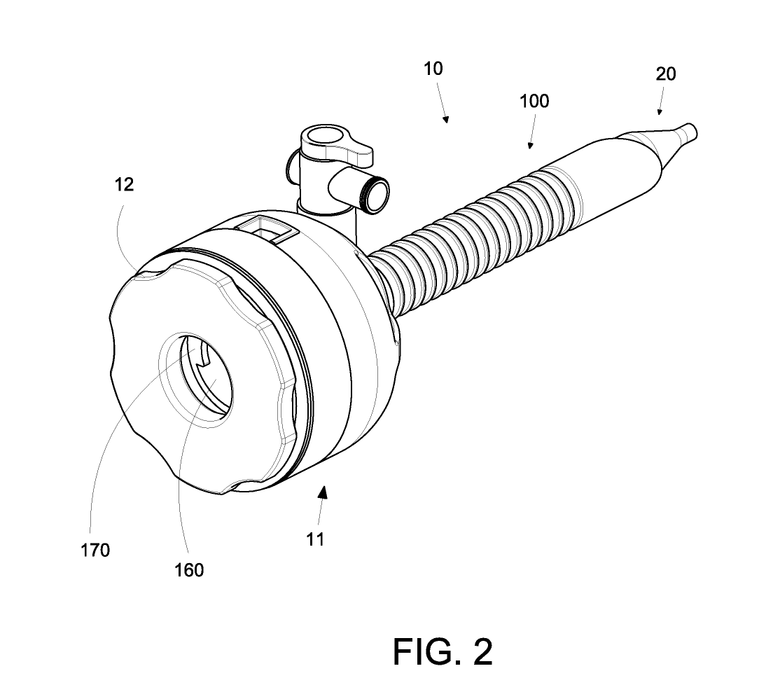

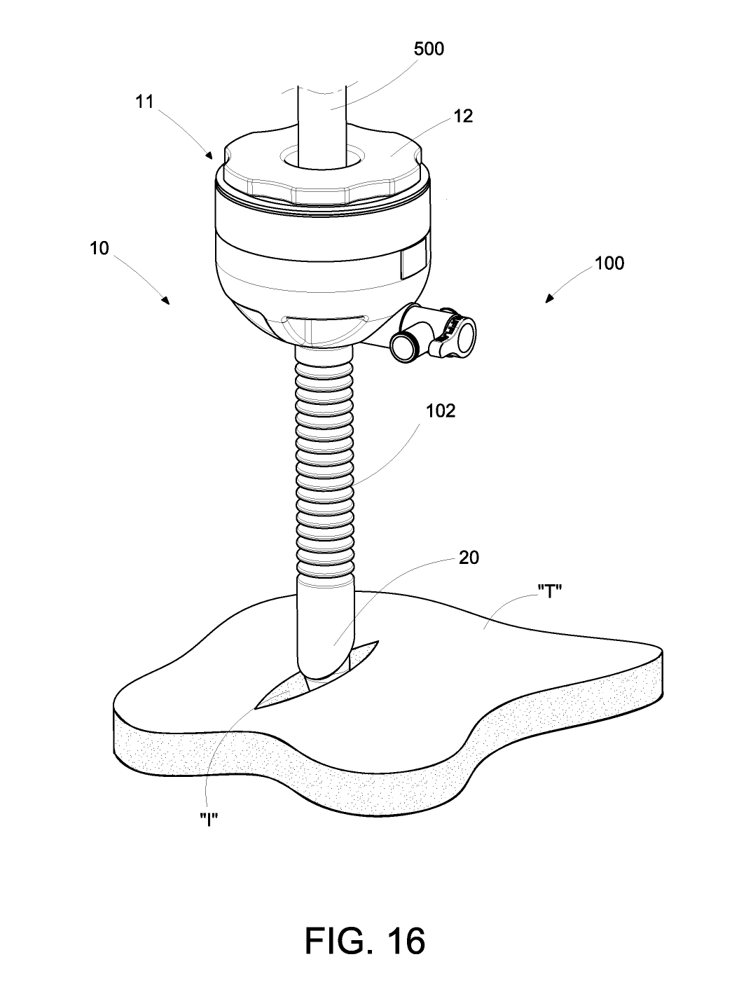

The obturator housing 12 of the obturator assembly 11 includes an opening 160 (see FIG. 2) and a scope retention member 170 (see FIG. 2) adjacent the opening 160. The scope retention member 170 is fabricated, for example, from an elastomeric material. The scope retention member 170 is adapted to engage the outer surface of the endoscope in frictional engagement therewith to assist in retaining the relative positioning of the endoscope 500 (see FIG. 16) within the obturator assembly 11.

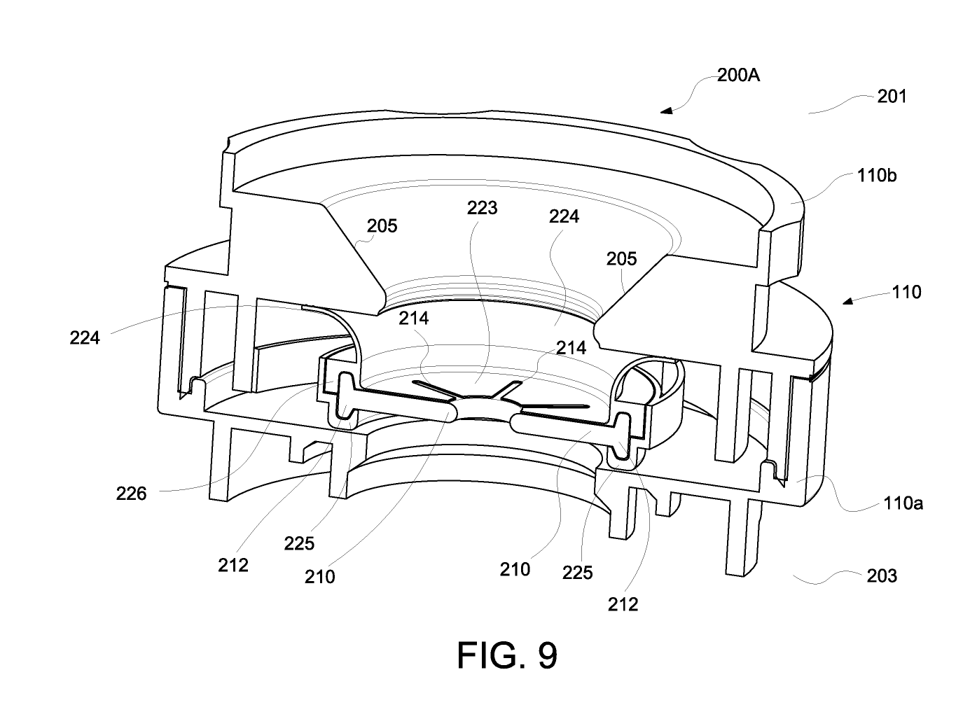

The cannula assembly 100 of the obturator 10 includes a translucent elongated portion 102 (see FIG. 1), defining a longitudinal axis “B-B,” and a cover 110. The cover 110, which includes a proximal housing component 110a and a distal housing component 110b (see FIG. 3), encloses a seal 210 (see FIGS. 9-11) and, in certain embodiments, a zero-closure seal 150 (see FIGS. 3, 7, and 8). More particularly, the proximal housing component 110a encloses the seal 210, and the distal housing component 110b encloses the zero-closure seal 150. Additionally, the cover 110 may be referred to as a seal assembly 110 having an upper housing member 110b and a lower housing member 110a.

The cover 110 is configured to mechanically engage a proximal portion of the elongated portion 102 and helps maintain the seal 210 and the zero-closure seal 150 therein. The cover 110 also includes a pair of notches 126 (FIG. 6) that are configured to be mechanically engaged by a pair of latches 19 disposed on the obturator assembly 11 (see FIG. 8). The selective engagement between the latches 19 and the notches 126 enables a user to selectively lock and unlock the obturator assembly 11 to and from the cannula assembly 100.

With further regard to the cover 110, FIGS. 4-8 illustrate additional features of the two-piece cover 110. The proximal housing component 110a (see FIG. 6) defines an inner wall 1112 and an outer wall 1114, which is disposed radially outwardly of the inner wall 1112. The inner wall 1112 defines a central passage 1116, which is dimensioned to receive a surgical instrument 500 therethrough (see FIG. 16).

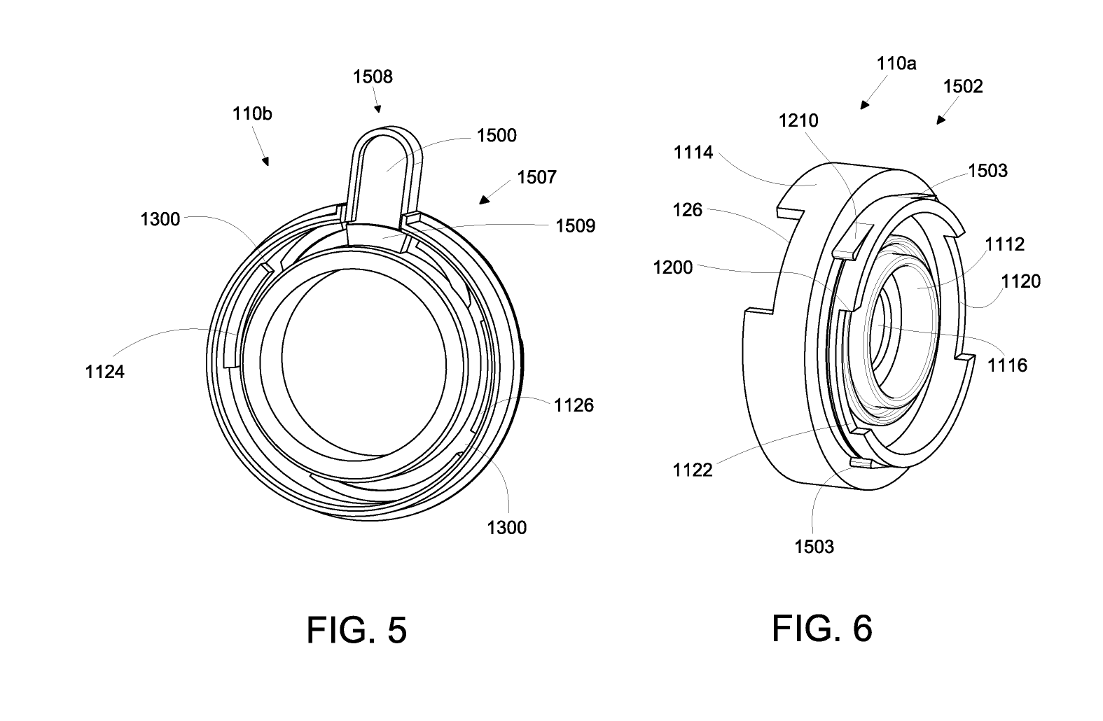

The outer wall 1114 defines first and second annular recesses 1120, 1122 adjacent its distal end (see FIG. 3). The recesses 1120, 1122 receive corresponding structure, i.e., annular lips 1124, 1126 of the distal housing component 110b (see FIG. 5) to facilitate connection of the two components. More particularly, a distal portion of the outer wall 1114 of the proximal housing component 110a includes a pair of ramps 1200, each of which is configured to engage a threaded portion 1300 and/or annular lips 1124, 1126 of the distal housing component 110b. Thus, coupling of the proximal housing component 110a and the distal housing component 110b is thereby affected through alignment and rotation of the components. Additionally, the proximal housing component 110a includes a stop 1210 adjacent each ramp 1200, which limits the rotational movement of the distal housing component 110b with respect to proximal housing component 110a.

A rotation prevention mechanism 1500 (see FIG. 5) is included to prevent inadvertent relative rotation and thus potential decoupling of the proximal housing component 110a and the distal housing component 110b. The rotation prevention mechanism 1500 includes a first component 1502 and a second component 1508. As shown, the first component 1502 consists of a finger 1503 that is integrally formed with a circumferential edge of the proximal housing component 110a. The second component 1508 of the rotation prevention mechanism 1500 is a tab 1507 that is integrally formed with a circumferential edge of the distal housing component 110b. The tab 1507 includes a radially outward user-actuatable portion 1508 and a radially-inward locking portion 1509. The tab 1507 is configured for resilient movement relative to the distal housing component 110b about its point of attachment thereto, such that its user-actuatable portion 1508 is moveable distally relative to the circumferential edge of the distal housing component 110b.

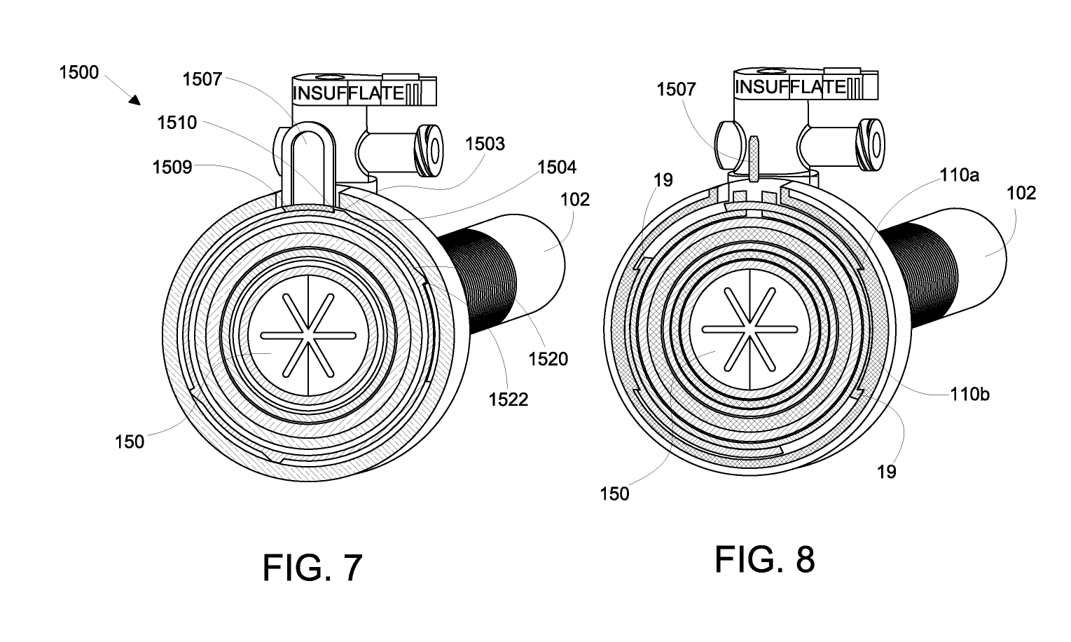

In this way, as the proximal housing component 110a is rotated in a first direction (i.e., clockwise when FIG. 7 is viewed from above), a ramped surface 1504 (see FIG. 7) of the finger 1503 engages the locking portion 1509 of the tab 1507. Continued rotation of the proximal housing component 110a causes the finger 1503 to exert a force directed in the radially outward direction on the locking portion 1509 of the tab 1507. The radially outward force is sufficient to cause the tab 1507 to move radially outward relative to the circumferential edge of the distal housing component 110b about its point of attachment thereto, from its first, biased position towards its second position. Additionally, the radially outward force causes the user actuatable portion 1508 of the tab 1507 to move distally. After a predetermined amount of rotation of the proximal housing component 110a, the finger 1503 passes the tab 1507, and causes the locking portion 1509 of tab 1507 to move back to its first, biased position and adjacent with a perpendicular surface 1510 of the finger 1503 (see FIG. 7). When the tab 1507 is in its first position, the proximal housing component 110a is effectively prevented from counter-clockwise rotation with respect to the distal housing component 110b.

Additionally, when sufficient rotation of the proximal housing component 110a causes the finger 1503 to pass the tab 1507, a protrusion 1520 of the proximal housing component 110a contacts a stop 1522 of the distal housing component 110b, thus effectively preventing additional clockwise rotation between the proximal housing component 110a and the distal housing component 110b (see FIG. 7). Accordingly, during the annular orientation of the proximal housing component 110a and the distal housing component 110b that is illustrated in FIG. 7, both directions of rotation of the proximal housing component 110a are effectively prevented, and thus the proximal housing component 110a is rotationally fixed with respect to the distal housing component 110b.

In this manner, the rotation prevention mechanism 1500 automatically prevents the proximal housing component 110a from inadvertently rotating relative to, and thus inadvertently becoming disconnected from, the distal housing component 110b once the proximal housing component 110a reaches this locked position.

To remove the proximal housing component 110a from the distal housing component 110b, a user exerts a force on the tab 1507 directed in a distal direction. A sufficient amount of distally-directed force causes the user actuatable portion 1508 of the tab 1507 to move distally relative to the circumferential edge of the distal housing component 110b about its point of attachment thereto until the locking portion 1509 of the tab 1507 is located radially outward of the finger 1503. In this position, the proximal housing component 110a is no longer prevented from rotating, but rather is free to rotate, in a second direction (i.e., counter-clockwise when FIG. 7 is viewed from above) relative to the distal housing component 110b. In this manner, the rotation prevention mechanism 1500 provides a selectively actuatable mechanism that, when actuated, enables a user to rotate, and, thereby, disconnect the proximal housing component 110a from the distal housing component 110b.

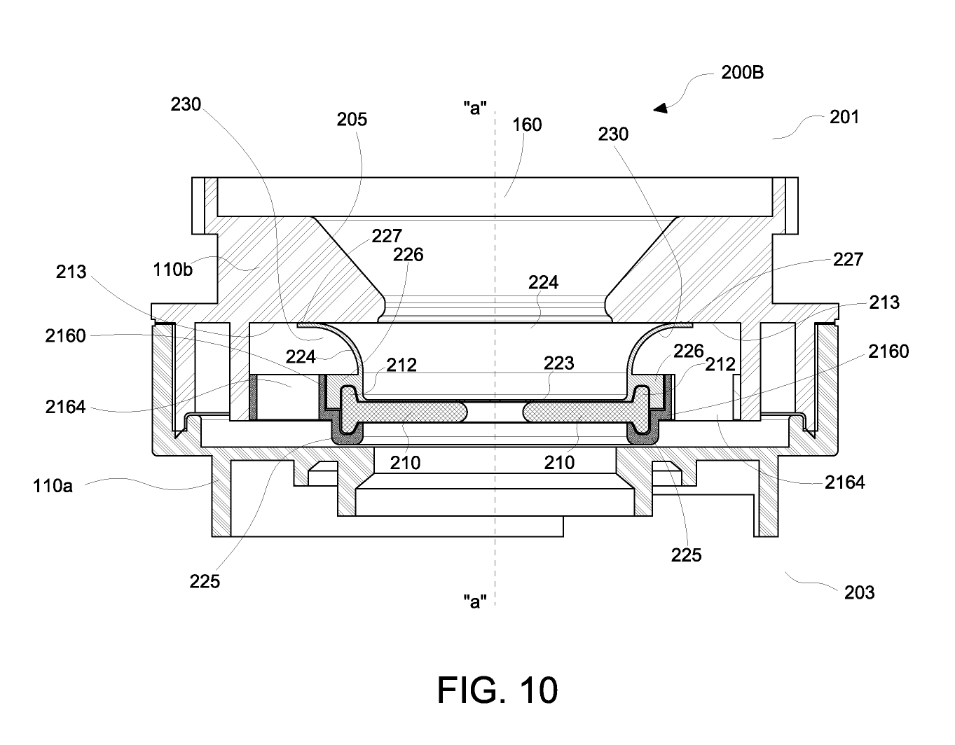

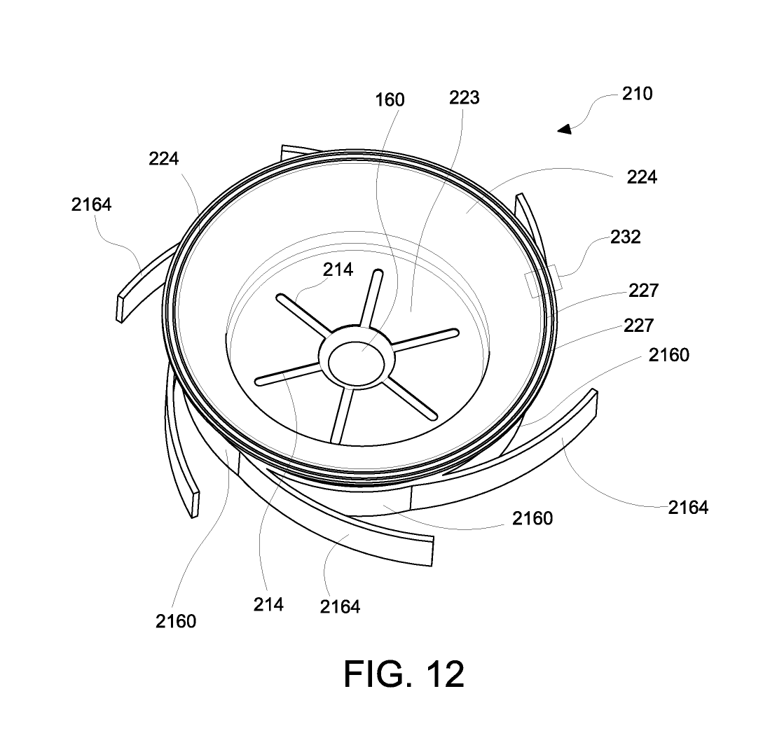

With reference to FIGS. 9-12, top view 200A or side view 200B of the seal access device includes seal assembly 110 (or cover 110) and longitudinal opening 160 extending through seal assembly 110. Seal assembly 110 defines central seal housing axis “a-a.” (See FIG. 10). Seal assembly 110 incorporates upper and lower housing components 110b, 110a which, when assembled together, form the seal assembly 110. Assembly of upper and lower housing components 110a, 110b may be effected by any suitable means, as discussed above with reference to FIGS. 4-8. Certain components or elements of the cover 110 described above with reference to FIGS. 4-8 may be removed when describing the seal assembly 110 in FIGS. 9-12 for sake of simplicity. Thus, the seal assembly 110 of FIGS. 9-12 will be described in terms of the upper housing member 110b and the lower housing member 110a, and how such components secure a seal 210 therebetween.

Referring to FIGS. 9-12, seal assembly 110 defines proximal and distal ends 201, 203, respectively. Adjacent proximal end 201 is internal tapered wall 205 which extends radially inwardly toward seal housing axis “a-a” from proximal to distal end, i.e., tapered wall 205 has both longitudinal and radial components of direction, and terminates in internal annular wall 205. Annular wall 205 serves to restrict the internal dimension of longitudinal opening 160 to at least partially constrain lateral movement of a surgical instrument 500 (see FIG. 16) introduced through seal assembly 110 via opening 160.

Seal assembly 110 may be constructed of a plurality of different materials, including, but not limited to, polymeric, metallic, or elastomeric. Preferably, the components of seal assembly 110 are formed of a polycarbonate material such as ABS available from the General Electric Company. Seal assembly 110 may further include a handle which may be of any suitable ergonomic design. Moreover, seal assembly 110 may be used in conjunction with, or detachably mounted, to cannula assembly 100 such as those described hereinabove.

Seal assembly 110 includes seal 210 mounted within seal assembly 110 in suspended relation. Seal 210 is mounted within seal assembly 110 such that the seal 210 is secured by an upper seal holder 223 and a lower seal holder 225. Seal 210 may be an elastomeric seal. It is also noted that seal 210 includes an opening through which an instrument may be received and which provides a seal with the instrument. The upper seal holder 223 may include a plurality of equally spaced apart slits 214 on a top portion thereof (see FIG. 9). The seal 210 is secured or sandwiched between the upper seal holder 223 and the lower seal holder 225. The upper seal holder 223 includes an upper lip 224 (or upper seal lip 224) and a lower member 226. The lower member 226 of the upper seal holder 223 and the lower seal holder 225 cooperate to secure the distal end or outer end or outer periphery 212 of the seal 210. The outer periphery 212 has a width greater than the width of the seal 210. The upper lip 224 of the upper seal holder 223 is configured to engage the upper housing member 110b to create a seal between the seal 210 and the upper housing member 110b. The upper seal holder 223 is dimensioned and adapted to prevent passage of fluids through the seal assembly 110. The upper seal holder 223 may further include a channel on a lower end thereof for receiving and securing a distal end 212 of the seal 210.

The upper seal holder 223 may also function as a guard member for the elastomeric seal. The guard member is adapted and dimensioned to protect the seal 210 from being ripped by an instrument 500 inserted therethrough (see FIG. 16). Advantageously, the guard member features of upper seal holder 223 (the flexible portions of the upper seal holder 223 that are located circumferentially between the slits 214) may be integrally formed with the upper seal holder 223. When the upper seal holder 223 is constructed or formed as a guard member as shown in, e.g., FIG. 9, the manufacturing of the device may be simplified and component costs may be reduced.

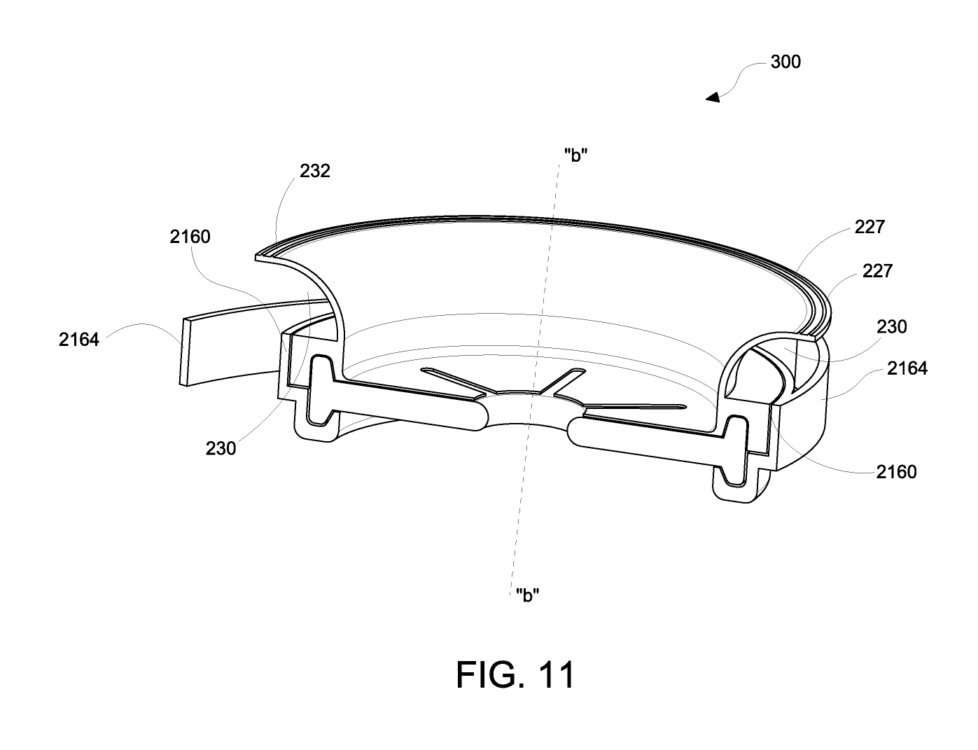

The upper seal lip 224 is in abutting relationship with the upper housing member 110b. The upper seal lip 224 has a substantially frusto-conical shape or has a substantially cup-shaped configuration. Stated differently, the upper seal lip 224 is adapted and dimensioned to extend outwardly with respect to the opening 160 to engage at least a portion of the upper housing member 110b. Thus, the upper housing member 110b defines an angular opening therethrough to facilitate angular reception of at least one surgical object or instrument. The upper lip seal 224 is constructed as a cup-shaped seal, where the top portion has a lip (or extension or projection) that acts as a wiper seal. The upper lip seal 224 is curved or bowed or arched or has a concave surface 230 (see FIGS. 10, 11), such that an upper portion 232 (see FIG. 11) of the upper lip seal 224 is substantially flat and perpendicular to the axis “b-b.” Stated differently, the upper portion 232 is substantially parallel to the seal 210. It is contemplated that the flat surface 230 directly contacts at least a portion of the upper housing member 110b. The upper lip seal 224 provides for the prevention of insufflation gases passing between the upper housing member 110b and the lower housing member 110a. The upper lip seal 224 is configured to hold the elastomeric seal 210 in place. By having only a portion of the upper seal holder 223 (i.e., upper lip seal 224) contact or engage a portion of the upper housing member 110b, low friction is created between seal assembly 110 and the seal 210, as well as a low cost of manufacturing for the seal assembly 110. For example, the flat surface 232 (see FIG. 11) of the upper seal lip 224 may only engage 10% of the surface area of the distal end 213 (see FIG. 10) of the upper housing member 110b. Of course, one skilled in the art may alter the size of the upper lip 224 to engage 5%, 15%, 20%, 25%, 30%, or any other percentage of the distal end 213 of the upper housing member 110b to reduce or increase the friction between the seal assembly 110 and the seal 210.

The upper seal lip 224 may include a plurality of ribs 227 (see FIG. 11) adapted and dimensioned to provide secure attachment to the upper housing member 110b, as depicted in view 300 of FIG. 11. The plurality of ribs 227 may be dual circumferential protrusions forming continuous dual concentric circles (see FIG. 11). Moreover, the upper seal lip 224 of the upper seal holder 223 may extend beyond the lower seal holder 226. The thickness of the plurality of ribs 227 may be equal to the thickness of the upper lip seal 224. However, it is contemplated that the thickness of the plurality of ribs 227 may be larger than or smaller than the thickness of the upper lip seal 224 based on the desired frictional relationship between the seal assembly 110 and the seal 210. For example, in one embodiment the thickness of the plurality of ribs 227 are twice the thickness of the upper lip seal 224.

It is envisioned that seal assembly 110 may be detachably connected to cannula assembly 100. Preferably, seal assembly 110 is dimensioned to be releasably mounted to cannula assembly 100. In this regard, it is appreciated that seal assembly 110 and the cannula assembly 100 may incorporate means for facilitating the releasable connection of seal assembly 110 to cannula assembly 100 including, e.g., an interference fit, bayonet coupling, screw arrangement, etc. on corresponding structure of the seal assembly 110 and the cannula housing 100, as described above with reference to FIGS. 4-8.

Moreover, the lower seal holder 225 includes a plurality of spaced apart and outwardly extending circumferential legs 2164 for providing self-centering of the seal 210. Thus, the housing component 110a may also be arranged to engage with or cooperate with or be associated with a plurality of spokes or spring elements 2164. Elements 2164 may aid in centering the housing component 110a, which in turn centers the seal 210 with respect to the seal housing axis “a-a.” (see FIG. 10) As illustrated, elements 2164 extend from the lower seal holder 225 to the side wall of the seal assembly 110. Thus, elements 2164 extend radially outwardly from an outer surface 2160 of the lower seal holder 225.

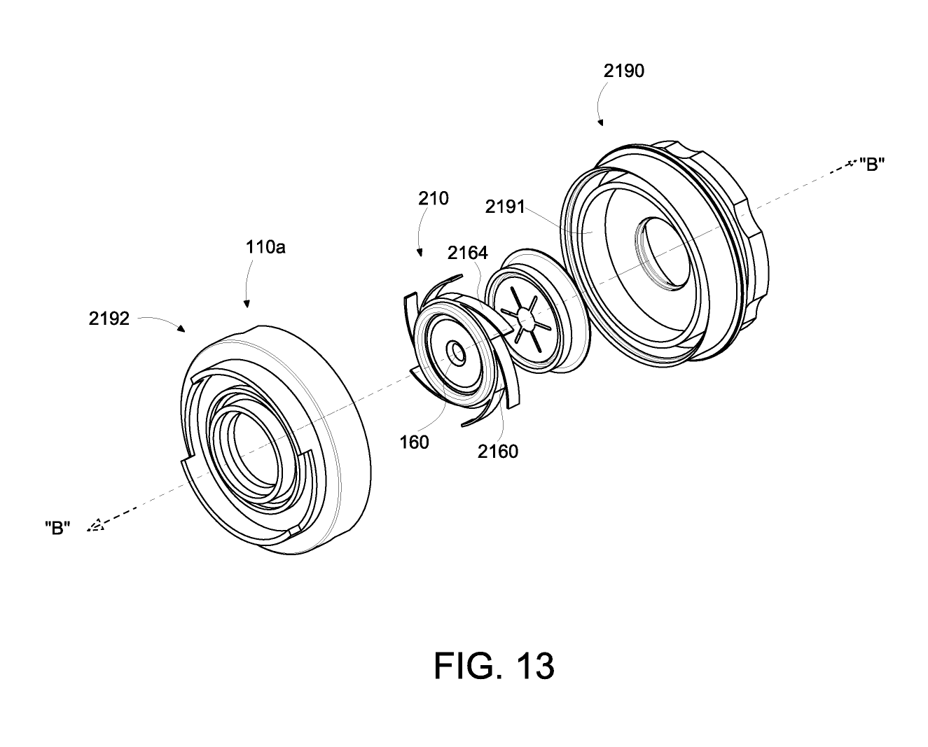

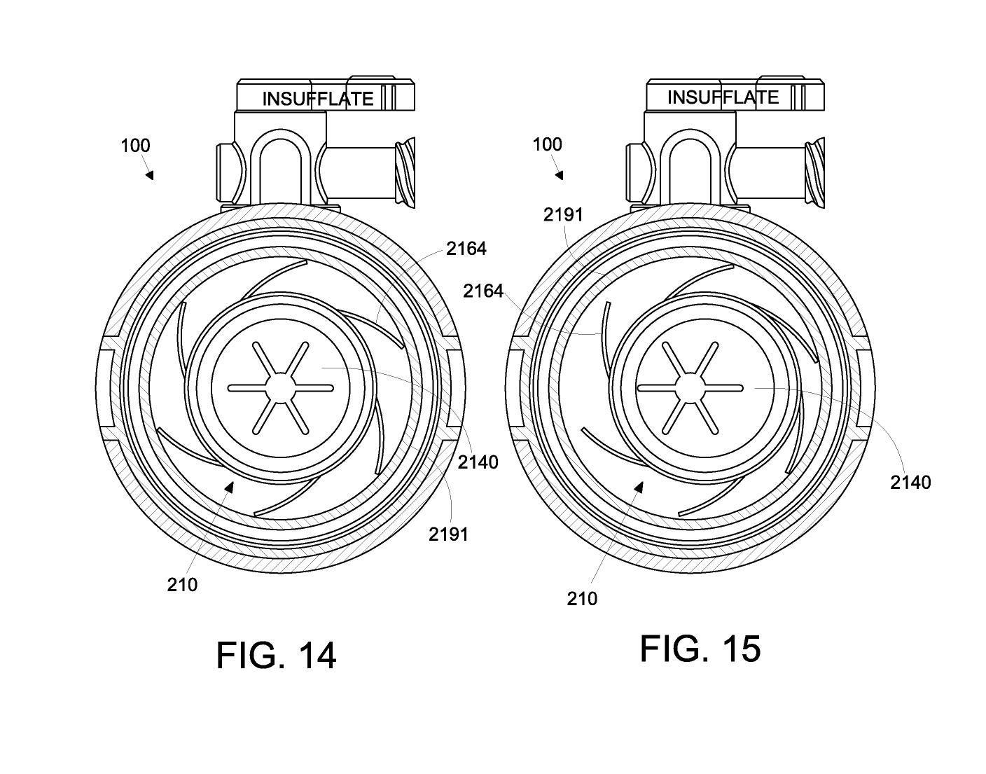

With reference to FIGS. 13-15, the seal 210 is positioned between a first portion 2190 and a second portion 2192 of the proximal housing component 110a, and is radially movable therein. The first portion 2190 and the second portion 2192 of the proximal housing component 110a are selectively engageable with each other via a snap-fit arrangement. Further, at least one spoke 2164 contacts an inner wall 2191 of the first portion 2190. The spokes 2164 are configured to help maintain the orifice 160 at or near the radial center within the proximal housing component 110a, and to help prevent free lateral movement of the septum seal 2140 within the proximal housing component 110a. That is, the spokes 2164 of the lower seal support 2160 bias the seal 210 toward the radial center of the proximal housing component 110a. Thus, upon removal of the obturator assembly 11 from the cannula assembly 100, the orifice 160 of the seal 210, which may otherwise move to an off-center location (as shown in FIG. 15), is urged toward the radial center of the proximal housing component 110a, such that the orifice 160 is in a centered location (see FIG. 14) for reception of a subsequently-inserted surgical instrument 500 (e.g., a surgical stapling device), as illustrated in FIG. 16 described below.

With reference to FIG. 16, in use, an initial incision “I” is made in tissue “T” (e.g., skin) by a surgical instrument (e.g., a scalpel). The obturator assembly 11 of the surgical obturator 10 is at least partially introduced within the cannula assembly 100 with the obturator member 14 (not shown) extending through the orifice 160 of the seal 210 and through the zero-closure seal 150. The assembled unit is positioned within the initial incision and against the target tissue, e.g., the abdominal lining. An endoscope 500 may be inserted through the obturator assembly 11 such that the endoscope may be retained at this relative position within the obturator assembly 11 by the scope retention member 170.

The optical member 20 (see FIG. 16) is manipulated relative to the tissue whereby the surgical instrument engages tissue and gently dissects or separates the tissue along a natural tissue plane to gain access to an underlying cavity in a non-traumatic fashion. During insertion, the tissue adjacent the optical member 20 is viewed with the endoscope 500. During advancement of the obturator 10, the endoscope 500 is utilized to view the path along which the obturator 10 is advanced to ensure that any underlying tissue or organ site is prevented from contact with the obturator assembly 11 and also to confirm entry within the body cavity. Once the obturator 10 is positioned at the desired location, the endoscope 500 may be used to monitor the desired surgical procedure being performed within the cavity. In the alternative, the endoscope 500 may be inserted into and secured in the obturator assembly 11 after the obturator assembly 11 has been positioned within tissue. The obturator assembly 11 may then be removed from the cannula assembly 100. Instruments may be introduced within the cannula assembly 100 to perform a surgical procedure.

Therefore, in summary, a seal assembly 110 includes an upper housing member 110b and a lower housing member 110a. Two seals may be positioned within the seal assembly 110. The first seal includes an upper seal holder 223 and a lower seal holder 225 for securing the second seal 210 in place. The upper seal holder 223 further includes an upper seal lip 224 that is configured to engage at least a portion of the upper housing member 110b to create a secure and low friction relationship between the seal 210 and the seal assembly 110. In some exemplary embodiments, the upper lip seal 224 may further include a plurality of ribs for further enhancing the secure relationship between the seal 210 and the seal assembly 110. The upper seal holder 223 may be referred to as a cup-shaped seal, where the upper portion (i.e., the upper seal lip 224) is in a substantially concave configuration extending beyond the lower seal holder 225. The upper lip seal 224 aids in reducing the friction or frictional relationship between the seal 210 and the seal assembly 110.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of presently disclosed embodiments. Thus the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure. As well, one skilled in the art will appreciate further features and advantages of the present disclosure based on the above-described embodiments. Accordingly, the present disclosure is not to be limited by what has been particularly shown and described, except as indicated by the appended claims.

What is claimed is:

1. A surgical access device, comprising:
a seal assembly having an upper housing member and a lower housing member configured to form a housing, the housing defining a central longitudinal axis and having a longitudinal passage for receiving at least one surgical object therethrough; and
a seal disposed within the seal assembly, the seal movably arranged within the housing, the seal including an elastomeric seal member having an opening for receiving and sealing with an instrument inserted therethrough, the elastomeric seal member arranged between an upper seal holder and a lower seal holder;
wherein the upper seal holder is a cup-shaped seal having an upper lip that engages at least a portion of the upper housing member to form a seal with the seal assembly.

2. The surgical access device according to claim 1, wherein the upper seal holder acts as a guard member for protecting the seal from being damaged by repeated insertion of the at least one surgical instrument therethrough.

3. The surgical access device according to claim 1, wherein the lower seal holder includes a plurality of spaced apart and outwardly extending circumferential legs for providing self-centering of the seal.

4. The surgical access device according to claim 1, wherein the upper housing member defines an angular opening therethrough to facilitate angular reception of the at least one surgical object.

5. The surgical access device according to claim 1, wherein the upper seal holder is dimensioned and adapted to prevent passage of fluids through the seal assembly.

6. The surgical access device according to claim 1, wherein the upper seal holder includes a channel on a lower end thereof for receiving and securing a distal end of the seal.

7. The surgical access device according to claim 1, wherein the upper lip of the upper seal holder includes a plurality of ribs adapted and dimensioned to provide a secure attachment to the upper housing member.

8. The surgical access device according to claim 7, wherein the plurality of ribs are dual circumferential protrusions forming continuous dual concentric circles.

9. The surgical access device according to claim 7, wherein the plurality of ribs are a series of discontinuous circular protrusions.

10. The surgical access device according to claim 1, wherein the upper lip of the upper seal holder extends beyond the lower seal holder.

11. A cannula assembly, comprising:
a cannula housing;
a cannula sleeve extending distally from the cannula housing; and
a seal assembly disposed in mechanical cooperation with the cannula housing, the seal assembly comprising:
a seal housing having an upper housing member and a lower housing member, the seal housing defining a central longitudinal axis and having a longitudinal passage for receiving at least one surgical object therethrough; and
a seal disposed within the seal assembly, the seal movably arranged within the housing, the seal including an elastomeric seal member having an opening for receiving and sealing with an instrument inserted therethrough, the elastomeric seal member arranged between an upper seal holder and a lower seal holder;
wherein the upper seal holder is a cup-shaped seal having an upper lip that engages at least a portion of the upper housing member to secure the seal within the seal housing.

12. The cannula assembly according to claim 11, wherein the upper seal holder acts as a guard member for protecting the seal from being damaged by repeated insertion of the at least one surgical instrument therethrough.

13. The cannula assembly according to claim 11, wherein the lower seal holder includes a plurality of spaced apart and outwardly extending circumferential legs for providing self-centering of the seal.

14. The cannula assembly according to claim 11, wherein the upper housing member defines an angular opening therethrough to facilitate angular reception of the at least one surgical object.

15. The cannula assembly according to claim 11, wherein the upper seal holder is dimensioned and adapted to prevent passage of fluids through the seal assembly.

16. The cannula assembly according to claim 11, wherein the upper seal holder includes a channel on a lower end thereof for receiving and securing a distal end of the seal.

17. The cannula assembly according to claim 11, wherein the upper lip of the upper seal holder includes a plurality of ribs adapted and dimensioned to provide a secure attachment to the upper housing member.

18. The cannula assembly according to claim 17, wherein the plurality of ribs are dual circumferential protrusions forming continuous dual concentric circles.

19. The cannula assembly according to claim 17, wherein the plurality of ribs are a series of discontinuous circular protrusions.

20. The cannula assembly according to claim 11, wherein the upper lip of the upper seal holder extends beyond the lower seal holder.
